(19) United States
(12) Patent Application Publication
(10) Pub. No.: US 2004/0152786 A1
(43) Pub. Date: Aug. 5, 2004

(54) Preventive or remedy for diseases caused by cerebrovascular disturbances

(76) Inventors: Bang Luu, Strasbourg (FR); Yoshihisa Kudo, Tokyo (JP); Masashi Yamada, Tokyo (JP); Masayuki Uchida, Odawara-shi (JP); Yukie Suma, Tokyo (JP); Hiroto Suzuki, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/478,477

(22) PCT Filed: May 24, 2001
(86) PCT No.: PCT/JP01/04358

(51) Int. Cl. A61K 31/12

(52) U.S. Cl. 514/690

(57) ABSTRACT

Provided is a preventive or remedy for diseases caused by a cerebrovascular disturbance, which comprises as an effective ingredient a cyclohexenone long-chain alcoholic compound represented by the following formula (1): [wherein, R¹, R² and R³ each independently represents H or CH₃ and X represents a C1₀₋₂₈ alkylene or alkenylene group]. The present invention makes it possible to effectively protect cerebral neurocytes from cerebrovascular disturbances such as cerebral infarction

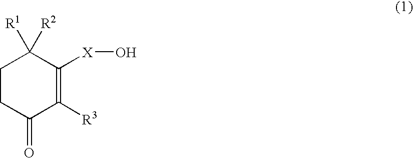

### TECHNICAL FIELD

The present invention relates to a preventive or remedy for diseases caused by cerebrovascular disturbances, which can protect cerebral neurocytes from cerebral ischemia caused by a cerebrovascular disturbance and markedly reduce the size of a lesion.

### BACKGROUND ART

The mortality due to cerebrovascular disturbances such as cerebral infarction in Japan has started to decrease since 1970 as a peak owing to improvement in the treatment during the acute phase. The incidence rate however does not seem to show a decrease. In consideration of the coming aged society, the number of patients suffering from them is presumed to rather increase from now on.

A cerebrovascular disturbance such as cerebral infarction, cerebral hemorrhage, subarachnoid hemorrhage or cerebral edema lessens the cerebral bloodstream, thereby causing cerebral ischemia. The cerebral ischemia increases the extracellular glutamic acid concentration, which excessively stimulates to the postsynaptic receptor for glutamic acid, and excessively increases the intracellular calcium ion concentration, causing cytopathy. Then, exfoliation of neurocytes occurs, leading to symptoms such as dementia. An improvement in the treatment of diseases induced by a cerebrovascular disturbance therefore depends on how to conduct an acute-phase treatment in order to protect neurocytes or how much its symptoms can be alleviated in the acute phase. The remedy employed clinically at present is an antiplatelet, anticoagulant or the like, which does not directly act to protect neurocytes (“Brain and Circulation”, 2, 13-17(1997)). It is known that re-perfusion of the blood after treatment with an antiplatelet or anticoagulant results in a stimulant such as NO damages cerebral neurocytes, thereby causing neurotic or mental disorders. There is accordingly a demand for the development of a medicament capable of controlling an excessive rise of the intracellular calcium ion concentration or of promoting discharge of the ion to directly protect neurocytes.

### DISCLOSURE OF THE INVENTION

An object of the present invention is therefore to provide a medicament capable of protecting cerebral neurocytes from cerebrovascular disturbances as described above.

With the foregoing in view, the present inventors carried out an extensive investigation. As a result, it has been found that the cyclohexenone long-chain alcoholic compound (WO99/08987) is capable of effectively protecting cerebral neurocytes from cerebrovascular disturbance and can markedly decrease the size of resulting lesions, leading to completion of the present invention.

The present invention provides a preventive or remedy for diseases caused by a cerebrovascular disturbance, which comprises as an effective ingredient a cyclohexenone long-chain alcoholic compound represented by the following formula (1):

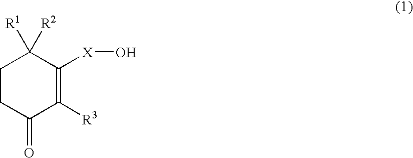

[wherein, R¹, R² and R³ each independently represents a hydrogen atom or a methyl group and X represents a linear or branched C₁₀₋₂₈ alkylene or alkenylene group].

The present invention also provides use of the cyclohexenone long-chain alcoholic compound for the manufacture of a preventive or remedy for diseases caused by a cerebrovascular disturbance.

The present invention further provides a method for the treatment of diseases caused by a cerebrovascular disturbance, whose method comrises administering the cyclohexenone long-chain alcoholic compound in an effective amount.

### BRIEF DESCRIPTION OF THE DRAWINGS

FIG. 1 is a graph illustrating comparison in an increase of the intracellular calcium ion concentration,

FIG. 2 is a graph illustrating the area (1) and volume (2) of the cerebral infarction lesion due to ischemia, and FIG. 3 is a graph illustrating the area (1) and volume (2) of the cerebral infarction lesion due to ischemic re-perfusion.

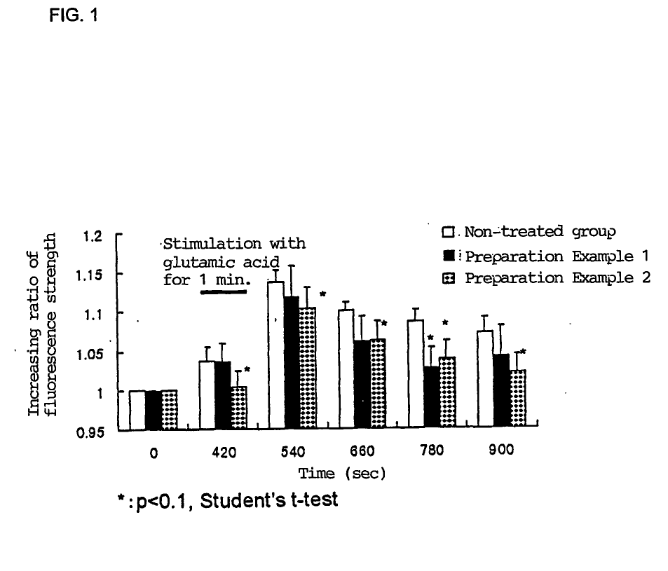

### BEST MODE FOR CARRYING OUT THE INVENTION

In the above-described formula (I), X represents a linear or branched C₁₀₋₂₈ alkylene or alkenylene group. The branched alkylene or alkenylene group contains, as a side chain, a C₁₋₁₀ alkyl group. Examples of the alkyl group as the side chain include methyl, ethyl, propyl, isopropyl, butyl, isobutyl, sec-butyl, tert-butyl, pentyl, isopentyl, neopentyl, tert-pentyl, hexyl, isohexyl, heptyl, octyl, nonyl and decyl groups. Among them, the methyl group is particularly preferred. The side chain is preferably substituted to a linear alkylene or alkenylene group (which means an alkene structure having at least one carbon-carbon double bond at the 3- and/or 7-position). For X, linear C₁₀₋₂₈ alkylene groups are preferred, with linear C₁₀₋₁₈ alkylene groups being particularly preferred. R¹, R² and R³ each independently represents a hydrogen atom or a methyl group, with the case where at least one of them represents a methyl group being more preferred.

The compound (1) of the present invention may exist as a pharmaceutically acceptable salt, or a solvate or hydrate thereof. The compound (1) of the present invention has various isomers and these isomers are also embraced by the present invention.

The compound (1) can be prepared, for example, in accordance with the following Process A or Process B.

[Process A]

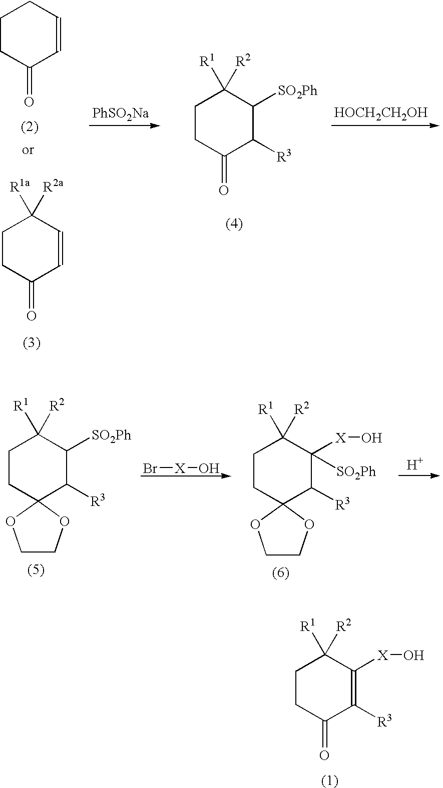

[wherein, R^{1a}, R^{2a} and R^{3a} each independently represents a hydrogen atom or a methyl group with the proviso that at least one of them represents a methyl group, Ph stands for a phenyl group and X, R¹, R² and R³ have the same meanings as described above].

Described specifically, the compound (1) can be obtained by reacting cyclohexenone (2) or methyl-substituted-2-cyclohexen-1-one (3) with a benzenesulfinic acid salt in the presence of an acid, reacting the resulting compound (4) with ethylene glycol, reacting the resulting ketal derivative (5) with a ω-halogenoalkanol or ω-halogenoalkenol, and subjecting the resulting compound (6) to an acid treatment to eliminate the protective group.

The methyl-substituted-2-cyclohexen-1-one (3) used here as a raw material is available by reacting methyl-substituted cyclohexanone with a trialkylsilyl halide in the presence of butyl lithium, followed by oxidation in the presence of a palladium catalyst.

The reaction of cyclohexenone (2) or methyl-substituted-2-cyclohexen-1-one (3) with a benzenesulfinic acid salt, for example, sodium benzenesulfinate is preferably effected in the presence of an acid such as hydrochloric acid, sulfuric acid or phosphoric acid at 0 to 100° C. for 5 to 40 hours.

The reaction of the compound (4) with ethylene glycol is preferably carried out in the presence of a condensing agent such as paratoluenesulfonic anhydride at 50 to 120° C. for 1 to 10 hours.

As the ω-halogenoalkanol to be reacted with the ketal derivative (5), a ω-bromoalkanol is preferably used. It is desired that the ketal derivative (5) is reacted with a ω-bromoalkanol in the presence of a metal compound such as butyl lithium under the low-temperature conditions.

The elimination of the phenylsulfonyl and ketal-protective groups from the compound (6) so obtained is preferably effected by reacting it with an acid such as paratoluenesulfonic acid.

[Process B]

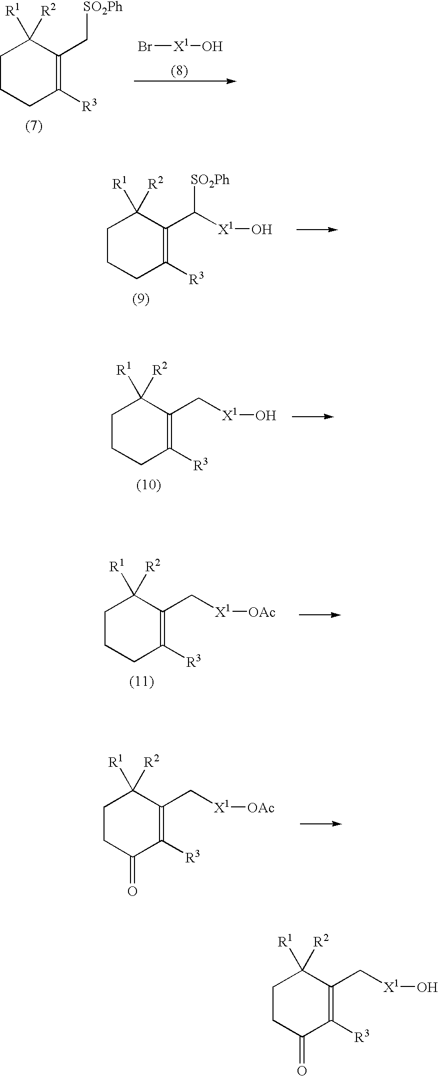

[wherein, X¹ represents a C₉₋₂₇ alkylene or alkenylene group, Ac stands for an acyl group and R¹, R², R³ and Ph have the same meanings as described above].

Described specifically, the compound (1a) can be obtained by reacting the compound (7) [available in accordance with, for example, Tetrahedron 52: 14891-14904,1996.] with ω-bromoalcohol, eliminating the phenylsulfonyl group from the resulting compound (9), protecting the hydroxy group of the resulting compound (10), oxidizing the resulting compound (11), and then eliminating the hydroxy-protecting group from the resulting compound (12).

The reaction of the compound (7) with the compound (8) is preferably conducted in the presence of a metal compound such as butyl lithium at low-temperature conditions.

The phenylsulfonyl group is eliminated from the compound (9) by reacting a phosphate salt in the presence of, for example, sodium amalgam.

As the hydroxy-protecting group of the compound (10), anacetyl group is preferred. The compound (10) is protected, for example, by reacting it with acetic anhydride.

The compound (11) is oxidized by reacting it with an alkyl hydroperoxide such as t-butyl hydroperoxide in the presence of a metal compound such as ruthenium trichloride.

The deprotection of the compound (12) is preferably conducted by hydrolyzing it in the presence of a base such as potassium carbonate.

The compound (1) is useful as an effective ingredient of a preventive or remedy for mammalian diseases (including human diseases) induced by a cerebrovascular disturbance such as cerebral infarction, cerebral hemorrhage, subarachnoid hemorrhage or cerebral edema.

No particular limitation is imposed on the administration route of the compound (1). Either oral or parenteral (intramuscular, subcutaneous, intravenous, suppository, or the like) administration can be adopted.

Oral preparations can be formulated into tablets, covered tablets, coated tablets, granules, capsules, solutions, syrups, elixirs, oil or aqueous suspensions in a manner known per se in the art after the addition of an excipient and if necessary a binder, a disintegrator, a lubricant, a colorant and/or a corrigent. Examples of the excipient include lactose, corn starch, sucrose, glucose, sorbitol and crystalline cellulose. Examples of the binder include polyvinyl alcohol, polyvinyl ether, ethyl cellulose, methyl cellulose, gum arabic, tragacanth, gelatin, shellac, hydroxypropyl cellulose, hydroxypropyl starch and polyvinyl pyrrolidone.

Examples of the disintegrator include starch, agar, gelatin powder, crystalline cellulose, calcium carbonate, sodium bicarbonate, calcium citrate, dextran and pectin; those of the lubricant include magnesium stearate, talc, polyethylene glycol, silica and hardened vegetable oil. As the colorant, pharmaceutically acceptable ones as additives can be used. Examples of the corrigent include cocoa powder, menthol, aromatic acid, peppermint oil, camphor and cinnamon powder. The tablet can also be used in the form of a coated tablet available by applying sugar coating, gelatin coating or the like to granules as needed.

Injections, more specifically, subcutaneous, intramuscular or intravenous injections are formulated in a manner known per se in the art by adding a pH regulator, buffer, stabilizer and/or preservative as needed. It is also possible to fill the injection solution in a vial or the like and lyophilize it into a solid preparation which is reconstituted immediately before use. One dose is filled in a vial or alternatively, multiple doses are filled in one vial.

For a human adult, the dose of the invention compound as a medicament usually falls within a range of from 0.01 to 1000 mg/day, with a range of from 0.1 to 100 mg/day being preferred. This daily dose is administered once a day or in 2 to 4 portions a day.

### EXAMPLES

The present invention will hereinafter be described by Examples, but it should be borne in mind that the present invention is not limited to or by these examples.

### Preparation Example 1

In accordance with the manner of Example 10 in International Patent Publication (WO99/08987), 3-(14-hydroxytetradecyl)-4-methyl-2-cyclohexen-1-one was prepared.

### Preparation Example 2

In accordance with the manner of Example 28 in International Patent Publication (WO99/08987), 3-(15-hydroxypentadecyl)-2,4,4-trimethyl-2-cyclohexen-1-one was prepared.

Test 1

Effects of the compound (1) for protecting neurocytes upon ischemia were tested. When the brain undergoes ischemia, the extracellular glutamic acid concentration increases, which excessively stimulates to the postsynaptic receptor for glutamic acid, and excessively increases the intracellular calcium ion concentration, causing cytopathy. Effects for controlling an increase in the calcium ion concentration in the neurocyte caused by the stimulation by glutamic acid were studied.

The primary culture of neurocytes taken from the hippocampus of a 18-day-old rat embryo was stained with 7.5 μM of fura-2/AM and then allowed to stand in a fluorescence microscope image processor. The neurocytes were stimulated with 1 mM of glutamic acid for 1 minute and a change in the intracellular calcium ion concentration caused thereby was measured. Five minutes before the stimulation by glutamic acid, the compound (1) was administered in an amount of 10⁻⁷ M. The change in the intracellular calcium ion concentration was measured by staining the neurocytes with fura-2/AM, a calcium ion fluorescence indicator. The fura-2/AM emits fluorescence when combined with calcium ions released by the stimulation by glutamic acid or stimulation by ischemia and its fluorescence strength is in proportion to the intracellular calcium ion concentration. The fluorescence strength was measured using a fluorescence microscope image processor (1×-70 [Olympus]+Argus 50 [Hamamatsu Photonics]) and a change in the intracellular calcium ion concentration was observed. The results are shown in Table 1.

In the group not treated with the compound (1), the fluorescence strength showed a gradual rise by the stimulation with glutamic acid. In spite of washing with a normal nutritive solution, the fluorescence strength was maintained at a high level, though decreasing a little.

In the group treated with 10⁻⁷ M of the compound (1) 5 minutes before the stimulation with glutamic acid, a rise in the fluorescence strength was suppressed at a level lower than that of the above-described treatment-free group. Its fluorescence strength lowered slightly after washing with a normal nutritive solution and returned to substantially the same level with that before the

Test 2

An influence of the cyclohexenone long-chain fatty alcoholic compound, which had been obtained in Preparation Example 2, on the area and volume of the infarct lesion caused by ischemia was studied using a middle-cerebral-artery permanently-occluded rat model.

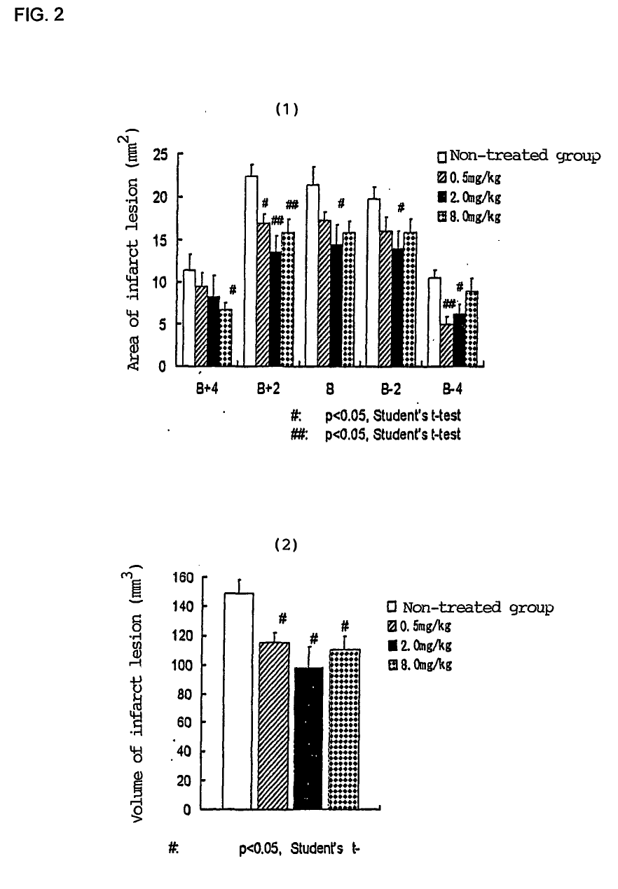

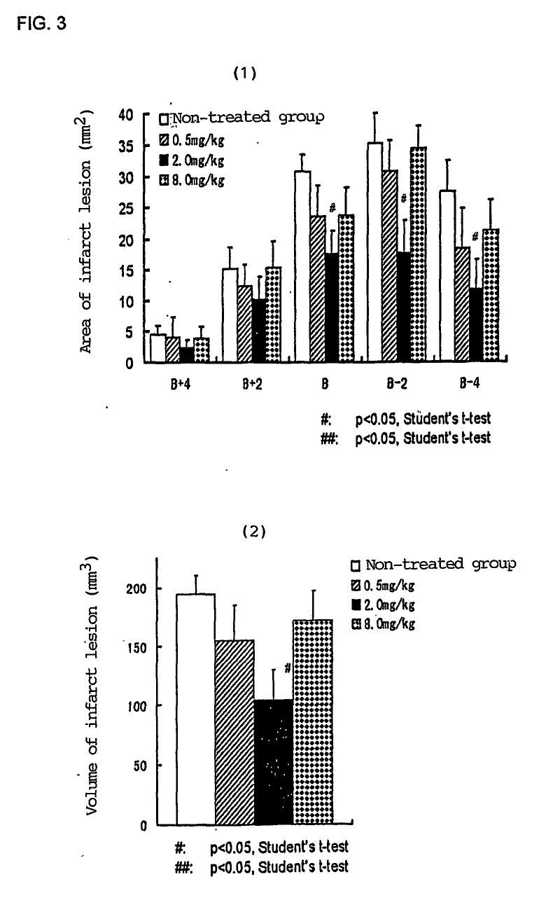

The middle cerebral artery of an 8-week-old rat was electrically coagulated and occluded by a bipolar coagulator (MICRO-3D [Mizuho Co., Ltd.]). The compound obtained in Preparation Example 2 was intraperitoneally administered in an amount of 0.3 mg/kg, 2 mg/kg and 8 mg/kg rightly after the occlusion of the middle cerebral artery, respectively. After 24 hours, the brain was enucleated and cut into slices of 2 mm thick. Each slice was stained with 1% 2,3,5-triphenyltetrazolium chloride (TTC), followed by the taking of photos. The photograph was analyzed and the area of the infarct lesion was measured. The volume of the infarct lesion was calculated from the below-described equation. The results are shown in FIG. 2.

V=a+2(b+c+d)+e

wherein,

a: infarct area (B+4) on the cross-section 4 mm forward from the forehead,

b: infarct area (B+2) on the cross-section 2 mm forward from the forehead,

c: infarct area (B) on the cross-section of the forehead,

d: infarct area (B-2) on the cross-section 2 mm backward from the forehead, and

e: infarct area (B-4) on the-cross-section 4 mm backward from the forehead.

As a result, a reduction in the area and volume of each infarct lesion was measured from the group administered with the compound (1). In particular, a marked brain protective action was recognized at a dose of 2 mg/kg.

Test 3

An influence of the cyclohexenone long-chain fatty alcoholic compound, which had been prepared in Preparation Example 2, on each of the area and volume of the infarct lesion was studied using an ischemic re-perfusion rat model.

The 8-week-old rats were occluded, at the middle brain artery thereof, with a nylon stopper for 2 hours, followed by intraperitoneal administration of the compound of Preparation Example 2 in amounts of 0.5 mg/kg, 2 mg/kg and 8 mg/kg, respectively, rightly before re-perfusion. After removal of the nylon stopper, re-perfusion was conducted. Twenty four hours after occlusion, the brain was enucleated and each of the area and volume of the infarct lesion was measured in a similar manner to Test 2. The results are shown in FIG. 3.

As a result, a reduction in the area and volume of each infarct lesion was measured from the group administered with the compound (1). Similar to Test 2, a marked brain protective action was recognized at a dose of 2 mg/kg.

Capability of Exploitation in Industry

The cyclohexenone long-chain fatty acid alcoholic compound (1) has cerebral neurocyte protective action by controlling an increase in the intracellular calcium ion concentration or promoting excretion of the intracellular calcium ion, thereby removing one of the factors of cytopathy; and decreasing the size of an infarct lesion caused by occlusion of the cerebral artery or ischemic re-perfusion. Accordingly, the compound (1) is useful as a remedy for diseases induced by a cerebrovascular disturbance such as cerebral infarction, cerebral hemorrhage, subarachnoid hemorrhage or cerebral edema.

1. A preventive or therapeutic for diseases caused by a cerebrovascular disturbance, which comprises as an effective ingredient a cyclohexenone long-chain alcoholic compound represented by the following formula (1): 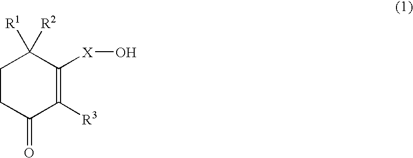
[wherein, R¹, R² and R³ each independently represents a hydrogen atom or a methyl group and x represents a linear or branched C₁₀₋₂₈ alkylene or alkenylene group]:

2. A preventive or therapeutic according to claim 1, wherein the cerebrovascular disturbance is cerebral infarction, cerebral hemorrhage, subarachnoid hemorrhage or cerebral edema.

3. Use of a cyclohexenone long-chain alcoholic compound represented by the following formula (1) for the manufacture of a preventive or therapeutic for diseases caused by a cerebrovascular disturbance: 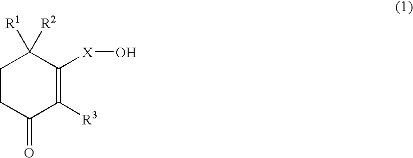
[wherein, R¹, R² and R³ each independently represents a hydrogen atom or a methyl group and X represents a linear or branched C₁₀₋₂₈ alkylene or alkenylene group].

4. The use according to clam 3, wherein the cerebrovascular disturbance is cerebral infarction, cerebral hemorrhage, subarachnoid hemorrhage or cerebral edema.

5. A method for the treatment of diseases caused by a cerebrovascular disturbance, which comprises administering a cyclohexenone long-chain alcoholic compound represented by the following formula (1) in an effective amount: 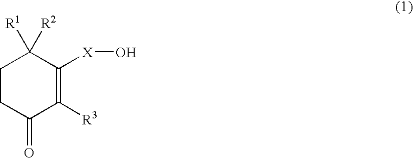
[wherein, R¹, R² and R³ each independently represents a hydrogen atom or a methyl group and X represents a linear or branched C₁₀₋₂₈ alkylene or alkenylene group].

6. The method according to claim 5, wherein the cerebrovascular disturbance is cerebral infarction, cerebral hemorrhage, subarachnoid hemorrhage or cerebral edema.
